(19) United States
(12) Patent Application Publication
(10) Pub. No.: US 2026/0207257 A1
(43) Pub. Date: Jul. 23, 2026

(54) SYSTEMS AND METHODS FOR PLANNING AND/OR NAVIGATING TO TREATMENT ZONES IN A MEDICAL PROCEDURE

(71) Applicant: INTUITIVE SURGICAL OPERATIONS, INC., Sunnyvale, CA (US)

(72) Inventors: Serena Wong, Los Altos, CA (US); Natalie Roel Ng, San Francisco, CA (US)

(21) Appl. No.: 19/139,253

(22) PCT Filed: Dec. 12, 2023
(86) PCT No.: PCT/US23/83510

§ 371 Date: Jun. 13, 2025

Related U.S. Application Data
(60) Provisional application No. 63/387,381, filed on Dec. 14, 2022.

(51) Int. Cl. A61B 34/10; A61B 34/00; A61B 34/20; A61B 34/30

(52) U.S. Cl. CPC A61B 34/10; A61B 2034/105; A61B 2034/107; A61B 2034/2061; A61B 2034/252; A61B 2034/301; A61B 2034/303

(57) ABSTRACT

Systems and methods are described for planning, and/or navigating during, a medical procedure. In one aspect, an expected trajectory of treatment tool that is extendable from a flexible elongate device is determined based on a pose of the flexible elongate device. A projected ablation zone is determined, in relation to a model representing an internal patient anatomy and a target lesion, based on the expected trajectory and an insertion distance of the treatment tool. A display device is caused to display a graphical user interface depicting the expected tool trajectory and the projected ablation zone. In another aspect, a position of a flexible elongate device is determined in relation to a model representing a target lesion. Trajectories along which the treatment tool can approach the target lesion are determined based on a position of the target lesion and the position of the flexible elongate device.

### CROSS-REFERENCE TO RELATED APPLICATIONS

This application claims priority to and the benefit of the filing date of provisional U.S. Patent Application No. 63/477,752 entitled “SYSTEMS AND METHODS FOR PLANNING AND/OR NAVIGATING TO TREATMENT ZONES IN A MEDICAL PROCEDURE,” filed on Dec. 14, 2022. The entire contents of the provisional application are hereby expressly incorporated herein by reference.

### FIELD

Disclosed examples relate to planning and/or navigating minimally invasive medical procedures and, more specifically, to systems and methods for planning, and/or guiding a user to, treatment zones in such procedures.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter, that can be inserted into anatomic passageways and navigated toward a region of interest within the patient anatomy.

Increasingly, robotically-assisted, minimally-invasive medical systems have provided graphical user interfaces (GUIs) to assist users (e.g., physicians) in navigating with flexible/steerable medical instruments so as to arrive at a desired treatment site (e.g., a lesion). For example, such robotically-assisted systems may be used to guide a mechanically steerable catheter to a lesion where treatment is to be performed using a tool that extends from the catheter. Due to recent advancements in imaging, modeling, and sensing techniques, GUIs provided by such navigating tools enable users to “see” where minimally invasive medical instruments are in relation to the patient's anatomy, and in relation to the target lesion. Moreover, planning tools can use models and/or images to enable users to plan the course of a procedure in advance. However, current navigating and/or planning tools can still require a substantial amount of trial and error during the actual procedure, with risks such as failure to treat (e.g., ablate) the entirety of a target lesion, damage to critical structures (e.g., organs) within the patient, and/or complications resulting from prolonged procedure times (e.g., due to delays caused by uncertainty and/or numerous treatment attempts).

### SUMMARY

The following presents a simplified summary of various examples described herein and is not intended to identify key or critical elements or to delineate the scope of the claims.

In some examples, a method for planning, or navigating during, a medical procedure includes obtaining, by one or more processors, a model representing an internal anatomy of a patient and a target lesion, and determining, by the one or more processors and based on a pose of a flexible elongate device, an expected tool trajectory of a treatment tool extendable from the flexible elongate device. The method also includes determining, by the one or more processors and based on the expected tool trajectory and an insertion distance of the treatment tool, a projected treatment zone in relation to the model, and causing, by the one or more processors, a display device to display a graphical user interface depicting (i) the expected tool trajectory, and (ii) the projected treatment zone, in relation to the model.

In other examples, a method for planning, or navigating during, a medical procedure includes obtaining, by one or more processors, a model representing a target lesion within a patient, and determining, by the one or more processors, a position of a flexible elongate device in relation to the model. The method also includes determining, by the one or more processors and based on a position of the target lesion and the position of the flexible elongate device, a plurality of trajectories along which a treatment tool extendable from the flexible elongate device can approach the target lesion to collectively provide treatment coverage across at least one dimension of the target lesion.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 depicts a graphical user interface that may be generated and displayed to a user, according to some examples.

FIGS. 2A-2C depict example scenarios in which a user operates the graphical user interface of FIG. 1, according to some examples.

FIG. 3 is a flow diagram of a method for planning, or navigating during, a medical procedure using a graphical user interface, such as the graphical user interface of FIG. 1, according to some examples.

FIG. 4 depicts an example projection of points representing a target lesion for purposes of determining suggested trajectories along which a treatment tool extendable from a flexible elongate device can approach the target lesion to collectively provide treatment coverage across at least one dimension of the target lesion, according to some examples.

FIG. 5 depicts an example representation of expected treatment coverage resulting from suggested trajectories, according to some examples.

FIG. 6 is a flow diagram of a method for planning, or navigating during, a medical procedure by determining trajectories, according to some examples.

FIG. 7 is a simplified diagram of a medical system in which techniques disclosed herein may be implemented, according to some examples.

FIG. 8A is a simplified diagram of a medical instrument system, including a flexible elongate device, which may be used in connection with the techniques disclosed herein, according to some examples.

FIG. 8B is a simplified diagram of a medical tool within the flexible elongate device of FIG. 8A, according to some examples.

FIGS. 9A and 9B are simplified diagrams of side views of a patient coordinate space including a medical instrument mounted on an insertion assembly, according to some examples.

Examples of the present disclosure and their advantages are best understood by referring to the detailed description that follows. It should be appreciated that like reference numerals are used to identify like elements illustrated in one or more of the figures, wherein showings therein are for purposes of illustrating examples of the present disclosure and not for purposes of limiting the same.

### DETAILED DESCRIPTION

In the following description, specific details are set forth describing some examples consistent with the present disclosure. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, components, and circuits have not been described in detail so as not to unnecessarily obscure aspects of the examples.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

This disclosure generally relates to systems and methods that facilitate user (e.g., surgeon or physician) planning of, and/or navigation during, a medical procedure such as, but not limited to, an endoluminal treatment procedure.

In one aspect, the systems and methods provide a graphical user interface (GUI) that enables a user to visualize and mark one or more treatment (e.g., ablation) sites/zones in relation to a displayed model of the patient's internal anatomy, at a pre-operative planning stage and/or intra-operatively during a procedure. The GUI depicts the anatomy model, including a target lesion (e.g., a targeted tumor, abscess, or other region of organ or tissue that has been created or otherwise affected by injury or disease), and depicts a flexible elongate device (e.g., catheter) at a planned or current/real-time pose within a lumen (e.g., lung airway) of the model. Based on the pose of the flexible elongate device, the system determines an expected trajectory of a treatment tool (e.g., an electroporation probe such as a needle, balloon, or other structure, or a probe that performs a different type of ablation, or an injection tool, etc.) extending from the flexible elongate device, and the GUI depicts the expected trajectory in relation to the model. The system then determines a projected treatment zone (e.g., a region of lesion and/or tissue that is expected to be ablated based on known characteristics and/or settings of ablation equipment) along that trajectory, at a particular virtual/potential insertion distance, and depicts the projected treatment zone via the GUI. If the tool is a manually controlled tool, the user may adjust the virtual insertion distance via a control provided by the GUI (e.g., a slider), and/or adjust the pose of the flexible elongate device (e.g., via controls during intra-operative navigation), until the projected treatment zone is at a desired location. Using the information (e.g., insertion distance) determined by interaction with the GUI, the user may manually adjust the tool and then apply the treatment. The user may then “mark” the projected treatment zone, e.g., using another control provided by the GUI. Marking the treatment zone may change the appearance (e.g., color, pattern, texture, etc.) of the zone, and fix the zone on the GUI such that further adjustment of the insertion distance does not move the marked treatment zone. In alternative examples, the insertion distance is not a virtual insertion distance, but rather an actual insertion distance of the treatment tool, e.g., as measured/detected by an insertion sensor of the flexible elongate device during the procedure. In these scenarios, the user might not need to note the desired insertion distance and manually adjust the tool, as this may be done automatically. In some examples, needle insertion may be automatically actuated as well.

In another aspect, the systems and methods determine an optimal or near-optimal set of trajectories for approaching a target lesion using a treatment tool (e.g., electroporation needle or other treatment probe) delivered via a catheter or other flexible elongate device. The trajectories may be determined by projecting points representing a target lesion onto a plane defined by a distal end of the flexible elongate device (e.g., a plane normal to a pointing direction of the flexible elongate device and located at its distal end, or a plane normal to a vector between a point at the distal end of the flexible elongate device and a centroid of the target lesion, etc.). In some examples, it is assumed that the user will want to sweep the treatment tool across a series of different device angles to span a largest dimension (major axis) of the target lesion relative to the current position of the distal end of the flexible elongate device. In these examples, the major axis of a shape defined by the projected points is identified, and a series of trajectories (e.g., suggested angles or axes) of the flexible elongate device is determined to span the target lesion within a plane defined by the major axis and another vector defined by the position or pose of the flexible elongate device (e.g., a plane defined by the major axis and a pointing direction of the flexible elongate device, or by the major axis and a vector between a point at the distal end of the flexible elongate device and a centroid of the target lesion, etc.). An initial trajectory and subsequent trajectories may be determined by assuming that a user will start at one extreme of an angle range for the flexible elongate device, and successively change the angle in, for example, only one direction until a dimension of the entire target lesion is spanned with any desired margin (e.g., rather than starting closer to a center point and progressively moving outward in opposite directions). The incremental change in trajectory/angle/axis may be determined based on the furthest extent of the target lesion from the flexible elongate device and a predetermined/desired amount of treatment zone overlap, to ensure that treatment zones overlap sufficiently at that furthest extent. Moreover, in some examples, the systems and methods determine a viewing angle for an intra-operative imaging device that would allow the intra-operative imaging device to capture optimal or near-optimal images of the procedure across all of the determined/suggested trajectories. For example, the determined viewing angle may be one that provides a two-dimensional image corresponding to a plane defined by the major axis of the projected points and a pointing direction of the flexible elongate device (or defined by the major axis and a vector defined between a point at the distal end of the flexible elongate device and a centroid of the target lesion, etc.). The viewing angle may be indicated to a user via a GUI to support manual reorientation, and/or the intra-operative imaging device may be automatically reoriented to provide the determined viewing angle.

The aspects, systems, and methods described herein may provide a number of improvements relating to the planning of, and/or navigation during, a medical procedure. For example, systems and methods disclosed herein may enable a user to more efficiently, accurately, and/or precisely treat (e.g., ablate) a target lesion. In particular, the disclosed systems and methods may provide more complete coverage of a lesion plus any desired margin, while reducing both the number of required treatment zones and the inadvertent treatment of critical structures (e.g., healthy organs or tissues) that are outside the desired margin. It will be understood that such improvements do not constitute an exhaustive list, and other improvements will be clear according to the various examples discussed herein.

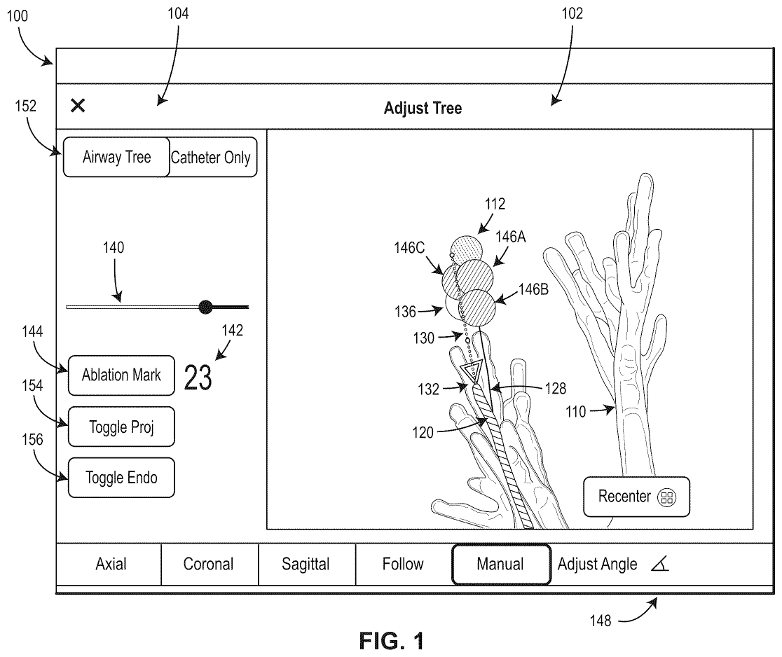

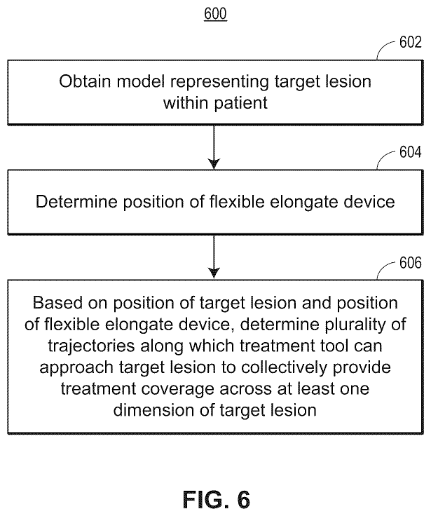

Referring first to FIG. 1, an example GUI 100 is provided to a user (or multiple users) to facilitate a robotically-assisted medical procedure. Specifically, the GUI 100 enables a user to visualize, consider, and decide upon actions for moving/guiding and/or operating a minimally invasive medical instrument (e.g., a flexible elongate device and a treatment tool extendable therefrom) within the anatomy of the patient. The flexible elongate device may be steerable using various controls (e.g., controls physically manipulated by the user, such as a trackball, scroll wheel, mouse, etc., or virtual controls on GUI 100 or another GUI). In the example of FIG. 1, the medical procedure is an endoluminal ablation procedure targeting a lesion within the patient's lungs, and the flexible elongate device is a catheter carrying/containing an ablation probe that is extendable from the catheter. The ablation probe (e.g., needle, balloon, and/or other structure) may perform ablation using radiofrequency ablation, microwave ablation, cryoablation, electroporation treatment, heat, or any other suitable ablation technique. Example systems and devices/tools for an endoluminal ablation procedure are discussed in more detail below with reference to FIGS. 7-9B. It is understood that a GUI similar to the GUI 100 may instead be used for other portions of a patient's anatomy (e.g., gastrointestinal procedures, cardiac procedures, etc.), and/or for medical procedures other than ablations, such as treatments involving injections into target lesions.

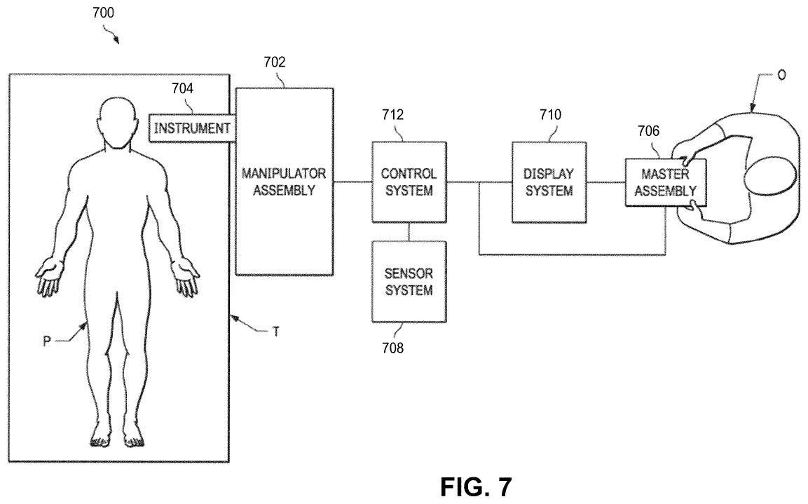

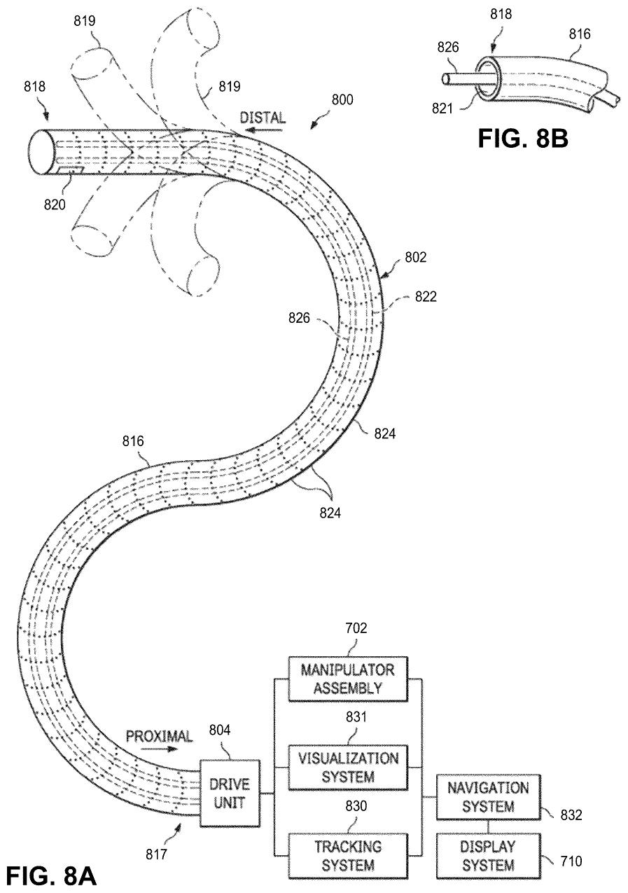

The GUI 100 may be generated by one or more processors of one or more computing devices and/or systems (e.g., one or more central processing units (CPUs) and/or one or more graphical processing units (GPUs)), which may in turn cause a display device (e.g., a dedicated or general-purpose monitor, or a head-mounted display unit, etc.) to display the GUI 100. For example, the processor(s) may render the GUI 100 and send the corresponding signals/data to the display device for display. For ease of explanation, descriptions below refer to operations by “the system,” which can be any suitable system (controller(s), etc.) or systems that (collectively) include the one or more processors. Specific examples of such systems, including systems or subsystems that may generate and present a GUI such as the GUI 100, are discussed below with reference to FIGS. 7 and 8A.

As seen in FIG. 1, the example GUI 100 generally includes a visualization portion 102 and a control portion 104. The visualization portion 102 depicts a model 110 of lung airways within the patient, with the model 110 including a visual representation 112 of a target lesion. For ease of explanation, the visual representation 112 is also referred to herein as simply target lesion 112. While referred to herein in the singular, it is understood that model 110 may consist of only a single model or may be an amalgam of multiple models. For example, the system may model the lung airways and the target lesion separately (possibly based on different imaging modalities), and register the two models with each other for appropriate relative placement within the visualization portion 102.

The system may generate the model 110 based on pre-operative imaging data and/or intra-operative imaging data. The pre-operative imaging data and/or intra-operative imaging data may be captured using any suitable imaging technology/modality or technologies/modalities, such as computed tomography (CT), cone-beam computed tomography (CBCT), magnetic resonance imaging (MRI), fluoroscopy, thermography, ultrasound, optical coherence tomography (OCT), thermal imaging, impedance imaging, laser imaging, nanotube X-ray imaging, and so on. In some examples, the system generates an initial model 110 based on pre-operative imaging data, and then verifies or updates the model 110 based on intra-operative imaging data (e.g., to correct for inaccuracies in the initial model 110 such as the configuration of the lung airways or the lesion size and/or position, possibly due to changes that occurred since the pre-operative images were captured). The process of updating the initial model 110 may include registering the intra-operative imaging data with the pre-operative imaging data and/or with the model 110 itself. In some examples, different imaging modalities are used to capture the pre-operative and intra-operative imaging data. For example, pre-operative imaging data may be captured using a CT imaging device, and intra-operative imaging data may be captured using a CBCT or fluoroscopy imaging device.

In examples where the GUI 100 is used for real-time navigation, the visualization portion 102 also depicts a visual representation 120 of a pose of the actual catheter within the patient's lung airways, in relation to the model 110. For ease of explanation, the visual representation 120 is also referred to herein as simply catheter 120. The system may position the catheter 120 in relation to the model 110 on the GUI 100 using any suitable technique, such as registering data from fiber-optic or electromagnetic shape sensor(s) (within the actual catheter) to the model 110, or registering camera image data to the model 110. Possible techniques for registering positions of portions of the actual catheter to the model 110, and/or to the imaging data used to obtain/generate the model 110, are discussed in more detail below in connection with FIG. 7. In alternative examples, the GUI 100 is used for pre-procedure planning, and the visual representation 120 is a planned or potential catheter pose (i.e., a pose of a virtual catheter) rather than an actual/real-time pose. In both navigation and planning examples, the GUI 100 may depict a planned route 128 to the target lesion 112, generated based on earlier, pre-operative plans (in navigation or planning examples) or as the route 128 is currently being generated (in planning examples).

The visualization portion 102 also depicts various dynamic graphical elements that can assist the user in approaching the target lesion with the ablation probe of the catheter. In particular, the visualization portion 102 depicts an expected tool trajectory (in this case, expected ablation probe trajectory) as a path 130 that starts at a distal end 132 of the catheter 120, and extends in the pointing direction of the catheter 120. The path 130 may be a dotted line (as shown) of any color or shade (e.g., white), or as a dashed line, a solid line, etc. In some examples, the path 130 extends up to a known/predetermined maximum insertion distance of the treatment tool (here, ablation probe), e.g., the maximum distance the tool can extend beyond a distal catheter end when fully extended. The system may limit the insertion distance entered/set by the user to the maximum extension distance, regardless of whether the length of the path 130 reflects or corresponds to that maximum distance.

The visualization portion 102 also depicts a projected treatment zone 136 (here, a projected ablation zone) that may be a sphere surrounding (centered at) a particular position along the path 130. The position and/or shape of the projected treatment zone 136 may depend on the properties of the treatment tool itself and how those properties affect the location and/or extent of the treatment zone in relation to the distal end of the catheter. Due to the (potentially) two-dimensional nature of the display, the zone 136 may appear as a circle on the GUI 100 at any given time, regardless of how the viewing perspective is changed. The system may place the projected treatment zone 136 at a position along path 130 that corresponds to a virtual insertion distance of the tool/probe of the catheter. In both real-time navigation examples and pre-operative planning examples, the virtual insertion distance may be entered by a user via a control 140 in the control portion 104. While FIG. 1 shows the control 140 as a virtual slider that the user can operate by touching the control 140 and moving his/her finger left or right, other control types are also possible (e.g., a virtual knob, or a field where a user may enter an insertion distance using a keyboard, etc.), or the GUI 100 may enable the user to change the virtual insertion distance by dragging and dropping the projected treatment zone 136 directly using his/her finger, etc. In other examples where the GUI 100 is used for real-time navigation, the system may place the projected treatment zone 136 at an insertion distance corresponding the actual, current (real-time) insertion distance of the treatment tool (here, ablation probe). For example, the catheter or treatment tool may include an insertion sensor that detects actual/real-time insertion distance, and sends data indicative of the insertion distance to the system for use in positioning the projected ablation zone 136 in relation to the model 110. Regardless of whether the insertion distance is an actual or virtual insertion distance, the control portion 104 may include an indication 142 of the (sensed or user-entered) insertion distance. A user may observe the indication 142 (e.g., “23” for 23 millimeters) and, when the user wishes to match the insertion distance of the actual flexible elongate device to what is displayed/indicated (and if the displayed value is within the capabilities of the catheter/treatment tool), the user can manually adjust a control or setting of the actual equipment/device to match the indicated value.

If the user touches or otherwise activates a control 144 in the control portion 104, the system changes the projected treatment zone 136 to a “marked” treatment zone, by changing the visual appearance and other properties of the projected treatment zone 136. Alternatively, the user may mark the treatment zone 136 by activating a control that serves another purpose, such as an ablation or biopsy button of equipment that includes or couples to the flexible elongate device. The user may mark a given projected treatment zone after actually performing the treatment (e.g., ablation) at the corresponding zone/region within the patient, for example (e.g., to provide an indication/reminder that the region no longer needs to be treated). In the example scenario of FIG. 1, the user has already operated the control 144 to mark three previous projected treatment zones, causing the system to change the zones into marked treatment zones 146A, 146B, and 146C. While FIG. 1 shows different interior patterns being used to distinguish projected treatment zones from marked treatment zones, the system may use any suitable technique to visually distinguish the two types of zones, such as using different colors (e.g., green for projected, red for marked), different shades (e.g., darker for marked and lighter for projected), and/or different patterns (e.g., as shown). Where projected and marked treatment zones overlap (and/or where marked treatment zones overlap each other), the system may use any suitable algorithm or rule for determining how to display overlapping portions (e.g., mix the colors of both, or always show green of a certain area/shape around a projected treatment zone, etc.).

In addition to changing the physical appearance of a projected treatment zone when the user marks that zone, the system may fix the position of the marked treatment zone in relation to the model 110, such that the position no longer moves (relative to model 110) responsive to further user input via the insertion distance control 140. In some examples, after a user marks a particular projected treatment zone, the next user input via the control 140 may cause a new projected treatment zone (similar to zone 136) to appear in the visualization portion 102 (e.g., at the same position the just-marked zone would have been located at if it had not been marked).

To create projected treatment zones at different desired positions relative to the target lesion 112 (and more generally, relative to the model 110), the user may use the control 140 to change the insertion distance, which causes the projected treatment zone 136 to responsively move a corresponding distance along the path 130, and/or the user may change the pose of the catheter 120. In real-time navigation examples, the user may change the pose of the catheter 120 by using physical or virtual controls to manipulate the pose of the actual catheter (e.g., by advancing the catheter and/or changing a pointing direction of the catheter). In pre-operative planning examples, the user may change the pose of the catheter 120 by using physical or virtual controls to simulate catheter movements.

To ensure that the actual target lesion is sufficiently covered/treated, the user may mark projected ablation zones (such as zone 136) until the target lesion 112 (plus any desired margin) is fully covered. The visualization portion 102 may visually depict the desired margin around the target lesion, if that margin is entered by the user or otherwise known to the system. To ensure that the actual target lesion is sufficiently covered in all dimensions/directions, the user may use a control 148 to change the virtual viewing angle at which the user observes the model 110 and catheter 120, which in turn causes the system to reposition the projected and marked zones 136, 146A-C accordingly (i.e., such that the zones are still in their same positions relative to the rotated/reoriented model 110 and catheter 120). In some real-time navigation examples, user adjustments to the viewing angle via the control 148 also cause an intra-operative imaging device (e.g., CBCT or fluoroscopy imaging device) to reorient to the same viewing angle.

The GUI 100 may also include additional controls to assist the user. In the example of FIG. 1, the GUI 100 includes a control 152 that the user may activate/manipulate to toggle between (1) displaying the lung airways and target lesion 112 of the model 110 along with the catheter 120 (i.e., as shown in the visualization portion 102 of FIG. 1), or instead (2) displaying only the catheter 120 (possibly still with the target lesion 112, but without the lung airways). The example GUI 100 also includes a control 154 that enables the user to toggle between showing or not showing the projected ablation zone 136, and a control 156 that enables the user to toggle an “endo” view on or off, where the endo view omits any current projected treatment zone (e.g., zone 136) and any/all marked treatment zones (e.g., zones 146A-C). Controls such as controls 152, 154, and/or 156 may help users make sense of what is depicted in the visualization portion 102, e.g., if the current display is too cluttered.

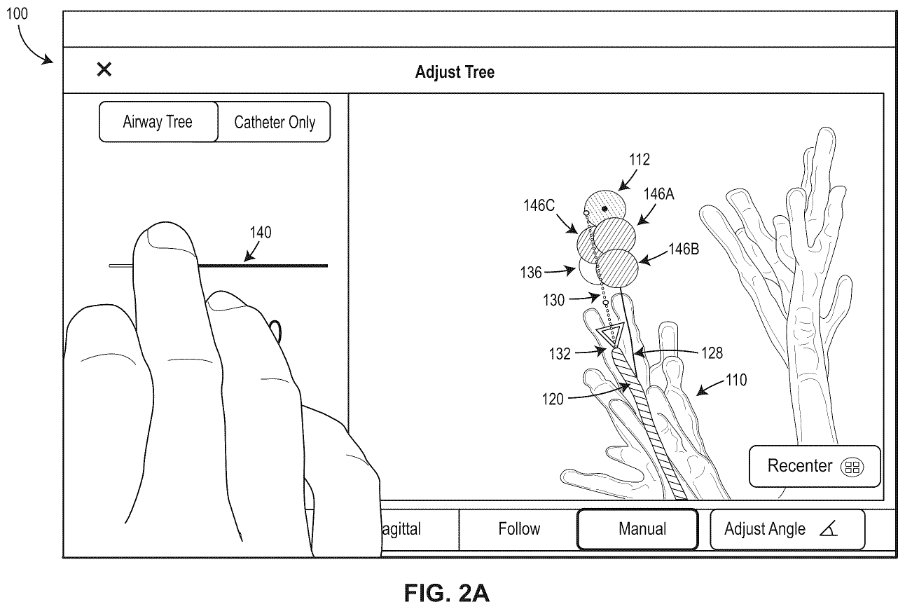

While FIG. 1 (like FIGS. 2A-C discussed below) depicts projected and marked treatment zones as spheres or circles (which may be partially obscured), it is understood that other shapes are possible (e.g., non-spherical ellipsoids, ellipses, etc.), based on known or expected characteristics of the treatment delivered by the ablation probe or other treatment tool. Additionally, the treatment zone in relation to the distal end of the catheter could be adjusted based upon the tool being used. In some examples, the system automatically determines/sets the size and extent (e.g., sphere radius) of each zone based on known or expected characteristics of the treatment delivered by the treatment tool, or based on current and/or entered equipment settings (e.g., ablation power and/or duration, or an injection amount, etc.) associated with the treatment tool. In some examples, the system automatically changes the color, shade, and/or pattern of a given treatment zone (e.g., only if a projected treatment zone, or possibly irrespective of whether the zone has yet been marked) based on one or more factors, such as whether the treatment zone overlaps any critical structures in the patient (e.g., organs or healthy tissues, as represented by the model 110, or as indicated by a user via the GUI 100, etc.). For example, the system may change a projected treatment zone 136 from green to white, and/or cause the zone 136 to flash, etc., if the insertion distance is set (or sensed) such that the projected ablation zone 136 would overlap (or come within some threshold distance of, etc.) a critical structure. Such features may assume default treatment parameters (e.g., default power and/or duration of ablation), or may take actual current settings into account.

In some examples, the GUI 100 enables the user to change the size of the projected treatment zone 136 (e.g., by performing a “drag” operation on a touchscreen displaying GUI 100, or via another virtual control of the GUI 100) before marking the zone 136. In response to these user changes to the treatment zone size, the system may automatically modify the power and/or duration of the treatment equipment (e.g., ablation device) to correspond to the size set by the user (e.g., higher power and/or longer duration for a larger size, and lower power and/or shorter duration for a smaller size). The system may limit such user changes based on a predetermined threshold (e.g., corresponding to pre-set maximum power and/or duration settings/values), and/or may cause the GUI 100 to display critical structures (organs, etc.) of the patient that should be avoided when the user is selecting/setting a treatment zone size.

In some examples, the system causes the GUI 100 to show, and/or causes the GUI 100 to restrict the user to reorienting the catheter 120 and/or treatment tool, to a permitted deployment range. In some planning examples, for example, the system and GUI 100 may only permit the user to alter the pose of the catheter 120 within some predetermined range. As another example, in some navigation examples, the GUI 100 may show the deployment range as a visual indicator surrounding the current, real-time position of the catheter 120 (e.g., surrounding distal end 132).

In some real-time navigation examples, the system causes the visualization portion 102 to automatically depict completed treatment zones after treatments (e.g., in response to each ablation being performed at a particular power and duration), without requiring the user to mark the zones. For example, when detecting that a user performs/triggers a treatment, the system may in response cause the visualization portion 102 to depict a marked treatment zone (e.g., zone 146A) in relation to the model 110. The marked/completed treatment zones may be set/indicated/depicted/etc. in the manner discussed above for user-marked treatment zones, and/or based on other factors. For example, the system may set the size and/or shape of depicted completed treatment zones based on various factors, such as treatment parameters (e.g., power and/or duration of the treatment), proximity to one or more critical structures of the patient, one or more known or expected characteristics (e.g., impedance) of tissue at the treatment site, and/or an actual (e.g., detected/sensed) size and/or shape associated with the treatment at the treatment site.

Example navigation and planning workflows are now described with reference to FIGS. 2A-C.

In an example ablation navigation workflow, the GUI 100 is used intra-operatively to create an ablation treatment plan during an ablation procedure, while a catheter is within a patient's anatomy. Initially, an imaging device (e.g., a CT imaging device) captures pre-operative imaging data of the patient, before the catheter is inside the patient. Based on the pre-operative imaging data, the system generates the model 110, and identifies the target lesion (based on user segmentation or automatic segmentation using the pre-operative imaging data) for inclusion in the model 110 as target lesion 112. When the catheter is within the patient, the system registers the catheter to the model 110. Optionally (e.g., before the catheter is in the patient), the user (or others) may use the model 110 to plan a route/path (e.g., as reflected by route 128) to the target lesion. In some cases, the user may perform a biopsy using the catheter, and the catheter may either be repositioned near the target lesion or left in place if already near the target lesion.

The user may then use the GUI 100 (e.g., after selecting an ablation mode) to intra-operatively plan an ablation. Initially (not shown in FIGS. 2A-C), the GUI 100 may display the model 110 with target lesion 112, and display the catheter 120 reflecting the real-time (e.g., sensed) pose of the actual catheter. The user may steer/drive the catheter to a position near the target (e.g., using route 128), and aim the catheter towards the target lesion (e.g., such that the distal end 132 of catheter 120 is pointed towards the target lesion 112). The system may capture additional imaging data using an intra-operative imaging device (e.g., a CBCT imaging device), and use the intra-operative imaging data to verify and/or update the pose of the target lesion 112, and possibly also catheter 120, within the model 110. The system may determine the catheter 120 and/or target lesion 112 poses from the intra-operative imaging data using segmentation and/or user identification.

The user can, with robotic assistance (e.g., as discussed below with reference to FIGS. 7-9B), alter the pose of the actual catheter to point towards the updated target lesion 112, if different from the initial lesion position. The system may use relative positions of the catheter and target lesion to update the target lesion position. The user may attempt to position the catheter so as to align the ablation probe trajectory (represented by path 130) along/within a plane of one of the axes of the target lesion. In particular, it may be beneficial for the user to align the trajectory within a plane that lies on a major axis of the target lesion (e.g., the axis along the longest lesion expanse/dimension), to allow the user to sweep the ablation probe through the plane (e.g., at a sequence of different angles) and thereby cover all or most of the target lesion with a relatively low number of treatments/ablations. The user may prefer to start at one edge of the target lesion 112, and change the angle in only one direction (with one or more insertion distances/treatments at each angle) until the entire target lesion 112 is spanned.

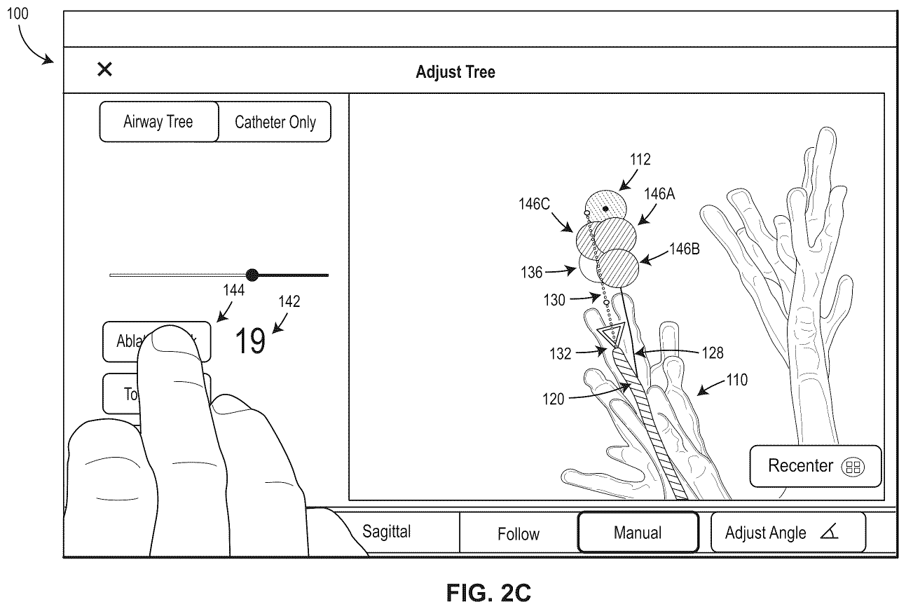

For each direction/angle of approach for the probe trajectory (with one potential direction/angle being represented by path 130), the user can (1) use the control 140 to select different insertion distances of the ablation probe (e.g., as shown in FIGS. 2A and 2B), (2) decide when the projected ablation zone provides appropriate coverage of the target lesion 112 (plus desired margin) and/or sufficient overlap with any previous marked ablation zones, (3) set the instrument according to the displayed insertion distance (and possibly desired power, duration, and/or other parameters) and perform an ablation, and (4) mark the treated/ablated zone as complete (e.g., as shown in FIG. 2C). The user can then withdraw the ablation probe into the catheter, steer the catheter to a new approach angle within the plane, and repeat one or more of these four steps. Subsequent approach angles may then be set, and one or more of the four steps repeated at each angle, until an entire dimension of the target lesion 112 is spanned along the major axis. In some examples, the system automatically determines the major axis of the target lesion 112 in the model 110, and/or determines a suggested viewing plane or angle that would provide an optimal or near-optimal view of a plane containing the major axis of the target lesion (e.g., such that the intra-operative imaging direction is orthogonal to that plane), and displays the major axis on the GUI 100 in relation to the model 110, and/or the suggested viewing plane or angle, to the user for guidance. Example calculations for determining the major axis are discussed in further detail below in connection with FIGS. 4-6. The user may have the goal of generating spheres (corresponding to marked/completed ablation zones) that cover the entire target lesion 112 and any desired margin. The user may use intra-operative imaging (e.g., a fluoroscopy device) to confirm the sweep/coverage, and to confirm insertion and retraction of the treatment tool.

In an example ablation planning workflow, the GUI 100 is used pre-operatively to create an ablation treatment plan for an ablation procedure. Initially, an imaging device (e.g., CT imaging device) captures pre-operative imaging data of the patient before the catheter is inside the patient. Based on the pre-operative imaging data, the system generates the model 110, and possibly also identifies the target lesion (based on user segmentation or automatic segmentation using the pre-operative imaging data) for inclusion in the model 110. The user may then use the GUI 100 to pre-operatively plan a path to the target lesion (e.g., route 128), including using the GUI 100 to identify an airway exit to the target lesion, as well as a deployment position (or parking location) of the catheter.

Initially, the GUI 100 may display the model 110 with target lesion 112, and the virtual catheter 120 with a probe trajectory positioned towards the target lesion 112. The virtual catheter 120 may be positioned/oriented in a pose that reflects the planned deployment position from the previous planning step, for example. The user may then alter the position or pose of the virtual catheter 120 so as to align the expected probe trajectory (path 120) with a major axis of the target lesion 112. In some examples, the GUI 100 enables (via touchscreen or other virtual control(s) such as virtual buttons) the user to drag the virtual catheter 120 to a new location in relation to the model 110, and/or to toggle the virtual catheter 120 between different poses.

The user can then use the control 140 to place the projected ablation zone 136 at the desired insertion position along the path 130 (e.g., as shown in FIGS. 2A and 2B), while monitoring the virtual insertion distance (via indication 142) to ensure that its value is within limits of the actual device/tool (e.g., in examples where the system does not automatically limit the virtual insertion distance to the allowed range). When satisfied, the user can use the control 144 to change the projected ablation zone 136 to a marked the ablation zone (e.g., similar to one of zones 146A-C), as shown in FIG. 2C. The user can repeat these steps of repositioning/reposing the virtual catheter 120, setting the virtual insertion distance, and marking the ablation zone as needed, e.g., until the target lesion 112 and any desired margin is sufficiently covered (e.g., using the sweeping technique discussed above in connection with the navigation workflow). Similar to the navigation workflow, the user may have the goal of generating spheres (corresponding to marked ablation zones) that cover the entire target lesion 112 and any desired margin. The user may then perform the actual ablation procedure according to the planned route 128 and marked ablation zones.

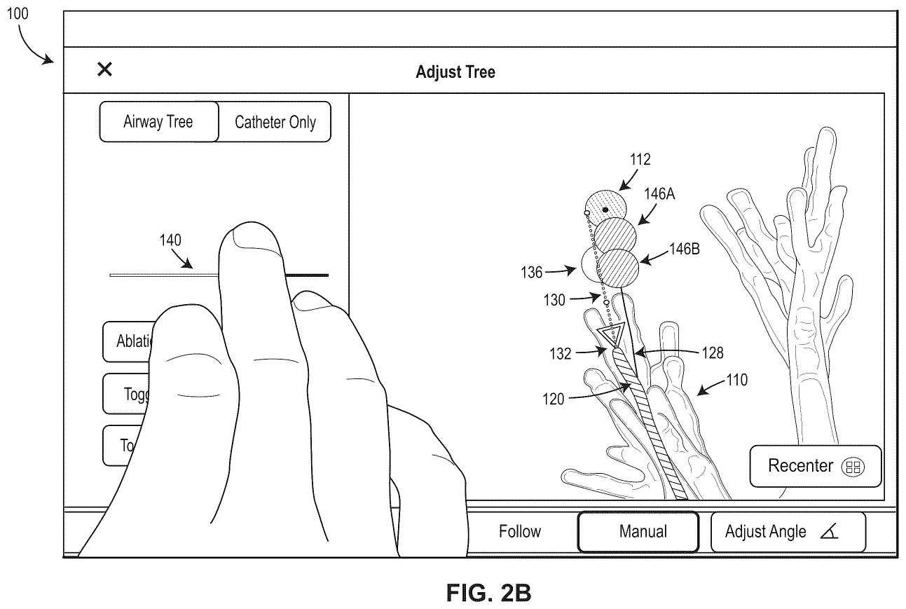

FIG. 3 depicts a method 300 for planning, or navigating during, a medical procedure using a graphical user interface, such as the graphical user interface of FIG. 1, according to some examples. The method 300 may be performed by one or more processors executing instructions stored in one or more computer-readable media (e.g., non-volatile memory), for example, such as various processor(s) of systems or subsystems discussed below in connection with FIGS. 7-9B.

At block 302, a model representing the internal anatomy of a patient and a target lesion (e.g., model 110) is obtained (e.g., generated from pre-operative and/or intra-operative imaging data as discussed above). Block 302 may include receiving the model, generating the model, or initially receiving or generating a model and then updating that model intra-operatively.

At block 304, an expected tool trajectory is determined based on a pose of a flexible elongate device (e.g., catheter). The tool may be a treatment tool (e.g., ablation probe, or injection tool, etc.) that can extend from (and retract within) the flexible elongate device. The flexible elongate device may be an actual device, with the pose being determined based on sensor or imaging data as discussed above, or being determined by receiving data that already indicates the pose in terms of the appropriate coordinate system. Alternatively, the flexible elongate device may be a virtual device (e.g., with the pose being controlled/determined by user inputs via a GUI).

At block 306, a projected treatment zone is determined in relation to the model, based on the expected tool trajectory that was determined at block 304 and an insertion distance of the treatment tool. The insertion distance may be a virtual insertion distance (e.g., a value entered by a user via a control such as control 140), or an actual insertion distance (e.g., as detected by an insertion sensor of the flexible elongate device, or as determined via intra-operative imaging).

At block 308, a display device is caused to display a GUI (e.g., similar to GUI 100) depicting the expected tool trajectory, and the projected treatment zone (e.g., zone 136), in relation to the model.

In some examples, the method 300 includes one or more additional blocks not shown in FIG. 3. For example, the GUI may depict the expected tool trajectory as a path that extends beyond a user-indicated/virtual insertion distance (e.g., path 130), and the method 300 may include an additional block in which the projected treatment zone is caused to move along the path on the GUI in response to user input via a virtual control (e.g., control 140), such as is shown in FIGS. 2A and 2B. The method 300 may also include limiting the user-indicated/virtual insertion distance to a known maximum insertion distance of the treatment tool.

As another example, the method 300 may include an additional block in which the GUI is caused to change the projected treatment zone to a marked treatment zone (e.g., one of zones 146A-C) that has a fixed position irrespective of further adjustments (e.g., further user adjustments) to the insertion distance.

As another example, the method 300 may include an additional block in which the GUI is caused to depict a completed treatment zone at the treatment site (in relation to the model), in response to detecting that a treatment is actually performed at that site (e.g., detecting that the user activates a control to perform the treatment). The method 300 may also include one or more additional blocks in which a size and/or shape of the completed treatment zone is determined based on proximity of the treatment site to one or more critical structures of the patient, one or more tissue characteristics of the patient at the treatment site, and/or an actual size and/or shape associated with the treatment at the treatment site (e.g., as detected using an impedance sensor or other sensor).

As another example, the method 300 may include a first additional block in which a major axis of the target lesion is determined (e.g., using projection techniques as discussed below), and a second additional block in which the GUI is caused to depict the major axis of the target lesion. The method 300 may also or instead include additional block(s) in which a viewing angle for viewing the medical procedure using an intra-operative imaging device is determined, and/or the GUI is caused to display such a viewing angle.

In other examples, the method 300 may include additional block(s) in which the GUI is caused (1) to adjust a size and/or shape of the projected treatment zone based on proximity of the projected treatment zone to one or more structures (e.g., critical structures) within the patient, (2) to adjust a color, shade, and/or pattern of the projected treatment zone based on overlap between the projected treatment zone and one or more structures within the patient, and/or (3) to adjust a size and/or shape of the projected treatment zone based on user input (and possibly also, in response to the user input, set a power and/or duration parameters of medical equipment to value(s) that correspond to the adjusted size and/or shape of the projected treatment zone).

In other examples, the method 300 may include additional block(s) in which (1) a maximum size of the projected treatment zone is limited based on a known limitation of the medical equipment, and/or (2) a suggested probe trajectory for a next treatment site is determined, and the GUI is caused to depict the suggested probe trajectory.

In one aspect of this disclosure, a system (e.g., the system discussed above in connection with GUI 100) can perform calculations to determine, based on a position of the flexible elongate device (e.g., catheter) in relation to the model (e.g., model 110), trajectories along which the flexible elongate device and its treatment tool (e.g., ablation probe) can approach the target lesion to collectively provide treatment coverage across at least one dimension of the target lesion.

In some examples, to determine the appropriate trajectories, the system accounts for not only the position of the target lesion, but also the position (e.g., parking location, corresponding to the distal end position), and possibly the pose (e.g., pointing direction), of the flexible elongate device. In an example navigation workflow, a user drives the flexible elongate device to the desired location, i.e., such that the distal end of the device is at the desired parking location near the target lesion. The user may consult intra-operative images (e.g., CBCT images) to confirm that the parking location and modeled target lesion location, as represented on a GUI (e.g., GUI 100), are correct. The system may update the locations (e.g., via x-y-z shifts) as needed if not correct, either automatically or in response to user inputs.

The system can then determine a “sweep” axis or direction based on the position or pose (e.g., the parking location and possibly also the pointing direction) of the flexible elongate device using a projection technique. One example projection technique is shown in FIG. 4, relative to a coordinate system 400. In FIG. 4, points 402 represent a target lesion, and location 404 represents the parking location of the flexible elongate device. Points 402 may be the representation of the target lesion in a model that was generated or otherwise obtained by the system (e.g., target lesion 112 of model 110), for example. Location 404 may be a center point of a circular distal end of a catheter (e.g., when the ablation probe or other treatment tool is fully retracted), for example, or another point at or near the distal end of the catheter.

To find/determine the appropriate axis/plane across which the treatment tool is to be swept, the system may project the points 402 onto a plane that includes the location 404. More specifically, the projection plane may be defined as a plane that passes through the location 404 and is orthogonal to a vector 406, where the vector 406 may be (1) a vector extending between the location 404 and another location associated with the points 402 (e.g., a centroid of the points 402), or (2) a vector extending from the location 404 in a pointing direction of the flexible elongate device. At least in the latter case, the system uses the pose of the flexible elongate device, rather than just the parking location/position, to determine the projection.

The system projects the points 402 representing the target lesion onto the plane, to determine projected points 410. The system can then determine a major axis 414 of a shape formed by the projected points 410. The system may determine the major axis 414 using principal component analysis or any other suitable technique. In some examples, the system approximates projected points 410 as an ellipse, and determines the major axis 414 as the major axis of the ellipse (and possibly also determines the orthogonal, minor axis 416).

The system may then use the major axis 414 to determine the trajectories. As noted above in connection with the GUI 100, it may be beneficial/efficient for the user to “sweep” the flexible elongate device and treatment tool across different angles within a plane, where the plane contains the major/long axis of the target lesion. Thus, the different trajectories may be different angles or axes of approach for the flexible elongate device within a “sweeping plane” defined by the identified major axis 414 and the vector 406. The system may cause a GUI (e.g., GUI 100) to display/indicate the trajectories (e.g., as angles or axes of approach). Additionally or alternatively, the system determines, and causes the GUI to indicate, an optimal (or near-optimal) viewing angle for an intra-operative imaging device (e.g., fluoroscopy or CBCT imaging device) to view the procedure while the treatment tool sweeps along the determined trajectories/angles. The viewing angle may be an angle that provides an imaging direction orthogonal/normal to the sweeping plane defined by the major axis 414 and the vector 406, for example. In some examples, the GUI includes a virtual control (e.g., button) that the user can use (select, activate, manipulate, etc.) to rotate the depicted model (e.g., model 110) to correspond to the viewing angle indicated on the GUI.

With the assistance of the information depicted on the GUI (e.g., trajectory angles and/or axes), the user can sweep the flexible elongate device and treatment tool using the determined/indicated trajectories, e.g., starting at one extreme edge of the target lesion and progressing to the opposite edge. In some examples, the system determines, and causes the GUI to indicate, an ordering of some or all of the determined trajectories. For example, the system may determine a suggested initial trajectory based on location 404, and further based on which of the projected points 410 is furthest from location 404 along or near the major axis 414 (e.g., to identify the most extreme sweeping angle in one direction). In some examples, the system also determines one or more subsequent trajectories to suggest. For example, the system may determine the one or more subsequent trajectories based on a predetermined/desired overlap of projected treatment zones (e.g., similar to zone 146) and the distance between location 404 and a furthest point of the target lesion from location 404. In some examples, the system may determine the distance to this “furthest” point of the target lesion by projecting at least some of the points 402 onto the vector 406, and then defining the furthest point as the point, from among the points projected onto vector 406, that is furthest from location 404.

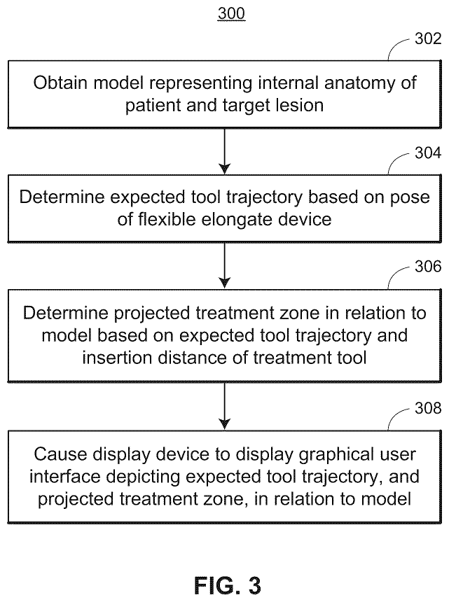

The system may use the distance of this furthest point from location 404 to ensure that the angles or axes of successive trajectories are close enough together to ensure the desired/predetermined amount of treatment zone overlap across the entire sweeping range of the flexible elongate device and treatment tool, even at the largest required insertion distances. FIG. 5 depicts, within a coordinate system 500, an example of resulting treatment zones 502 that are packed sufficiently close by the system so as to ensure full coverage of the target lesion, even at the largest insertion distances. In some examples and/or scenarios, this results in closer packing (more overlap) of treatment zones (e.g., spheres) as the distance from the parking location 404 decreases. In some examples, the system may also determine (and cause the GUI to display) insertion distances, at each of the determined trajectories, that ensure full coverage of the target lesion in at least a second dimension (e.g., in the direction of vector 404). In other examples, the user is fully responsible for setting insertion distances by observing the GUI (e.g., using the projected treatment zone and marked treatment zones described above in connection with FIGS. 1-3).

In some examples, in addition to determining trajectories relative to a first parking location, the system can determine/suggest trajectories relative to one or more other parking locations (e.g., if the target lesion is too large to be treated/covered completely from one parking location).

In some examples, the system automatically (e.g., in response to determining the trajectories, or after user confirmation via a virtual GUI control) causes a robotic system to perform treatments (e.g., ablations) at treatment zones corresponding to the determined trajectories and determined insertion distances along each trajectory.

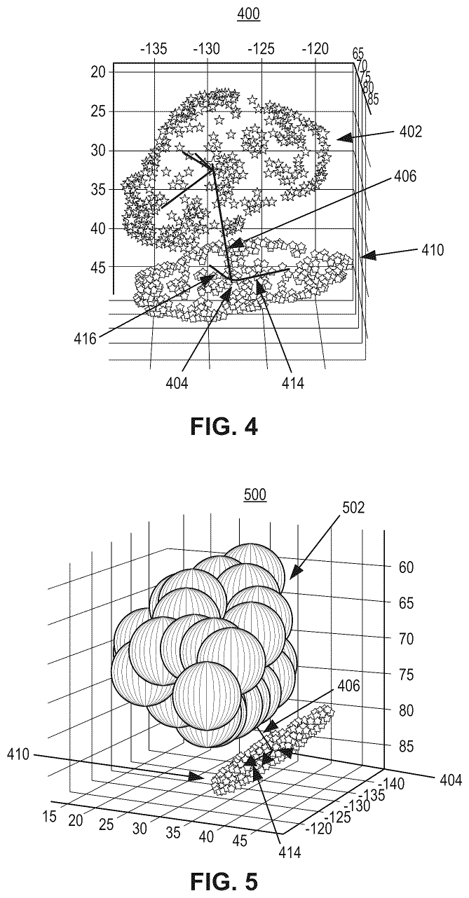

FIG. 6 is a flow diagram of a method 600 for planning, or navigating during, a medical procedure by determining treatment tool trajectories, according to some examples. The method 600 may be performed by one or more processors executing instructions stored in one or more computer-readable media (e.g., non-volatile memory), for example, such as various processor(s) of systems or subsystems discussed below in connection with FIGS. 7-9B.

At block 602, a model representing a target lesion (e.g., target lesion 112, possibly as part of model 110) is obtained (e.g., from pre-operative and/or intra-operative imaging data as discussed above). Block 602 may include receiving the model, generating the model, or initially receiving or generating a model and then updating that model intra-operatively.

At block 604, a position of a flexible elongate device is determined. Block 604 may include determining a position of the distal end of the flexible elongate device (“parking” location). In some examples, block 604 includes determining the pose of the flexible elongate device (e.g., the parking location and pointing direction of the distal end of the flexible elongate device). The flexible elongate device may be a catheter, for example, and contains a treatment tool (e.g., an ablation probe, or injection tool, etc.) that can extend from (and retract within) the flexible elongate device. The flexible elongate device may be an actual device (e.g., with the position or pose being determined based on sensor or imaging data as discussed above, or by receiving data that already indicates the position or pose in terms of the appropriate coordinate system), or a virtual device (e.g., with the position or pose being controlled/determined by user inputs via a GUI).

At block 606, a plurality of trajectories is determined based on the position of the target lesion and the position (and possibly also the orientation) of the flexible elongate device. The trajectories (e.g., represented as angles and/or axes that the flexible elongate device can be manipulated to form or align with) are trajectories along which the treatment tool of the flexible elongate device can approach the target lesion to collectively provide treatment coverage across at least one dimension (e.g., the major axis) of the target lesion. Block 606 may include using a projection technique, such as that described above in connection with FIG. 4, to determine a major axis and corresponding trajectories, for example. The trajectories may include a suggested initial trajectory and one or more suggested subsequent trajectories (e.g., as discussed above in connection with FIG. 4).

In some examples, the method 600 includes one or more additional blocks not shown in FIG. 6. For example, the method 600 may include an additional block in which a display device is caused to display the determined trajectories on a GUI (e.g., similar to GUI 100), in relation to the model. As another example, the method 600 may include a first additional block in which a viewing angle is determined for the medical procedure (e.g., based on the major axis 414 and vector 406 as discussed above), a second additional block in which a display device is caused to indicate the viewing angle to the user, and/or a third additional block in which an intra-operative imaging device is caused to reorient in accordance with the viewing angle (automatically or in response to a user input).

FIGS. 7-9B depict diagrams of a medical system that may be used for manipulating a medical instrument that includes a flexible elongate device according to any of the methods and systems described above, in some examples. For example, each reference above to the “system” may refer to a system (e.g., system 700) discussed below, or to a subsystem thereof.

FIG. 7 is a simplified diagram of a medical system 700, according to some examples. The medical system 700 may be suitable for use in, for example, surgical, diagnostic (e.g., biopsy), or therapeutic (e.g., ablation, electroporation, etc.) procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 7, the medical system 700 may include a manipulator assembly 702 that controls the operation of a medical instrument 704 in performing various procedures on a patient P. Medical instrument 704 may extend into an internal site within the body of patient P via an opening in the body of patient P. The manipulator assembly 702 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 702 may be mounted to and/or positioned near a patient table T. A master assembly 706 allows an operator O (e.g., a surgeon, a clinician, a physician, or other user) to control the manipulator assembly 702. In some examples, the master assembly 706 allows the operator O to view the procedural site or other graphical or informational displays. In some examples, the manipulator assembly 702 may be excluded from the medical system 700 and the instrument 704 may be controlled directly by the operator O. In some examples, the manipulator assembly 702 may be manually controlled by the operator O. Direct operator control may include various handles and operator interfaces for hand-held operation of the instrument 704.

The master assembly 706 may be located at a surgeon's console which is in proximity to (e.g., in the same room as) a patient table T on which patient P is located, such as at the side of the patient table T. In some examples, the master assembly 706 is remote from the patient table T, such as in in a different room or a different building from the patient table T. The master assembly 706 may include one or more control devices for controlling the manipulator assembly 702. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 702 supports the medical instrument 704 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 712). The manipulator assembly 702 may include a plurality of actuators (e.g., motors) that drive inputs on the medical instrument 704 in response to commands, such as from the control system 712. The actuators may include drive systems that move the medical instrument 704 in various ways when coupled to the medical instrument 704. For example, one or more actuators may advance medical instrument 704 into a naturally or surgically created anatomic orifice. Actuators may control articulation of the medical instrument 704, such as by moving the distal end (or any other portion) of medical instrument 704 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 704, such as for grasping tissue in the jaws of a biopsy device and/or the like, or may be used to move or otherwise control treatment tools (e.g., imaging tools, ablation tools, biopsy tools, electroporation tools, etc.) that are inserted within the medical instrument 704.

The medical system 700 may include a sensor system 708 with one or more sub-systems for receiving information about the manipulator assembly 702 and/or the medical instrument 704. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 704; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 704 or from some other location; and/or actuator position sensors such as resolvers, encoders, potentiometers, and the like that describe the rotation and/or orientation of the actuators controlling the medical instrument 704.

The medical system 700 may include a display system 710 for displaying an image or representation of the procedural site and the medical instrument 704. Display system 710 and master assembly 706 may be oriented so physician O can control medical instrument 704 and master assembly 706 with the perception of telepresence. In some examples, although the display system 710 and the master assembly 706 are depicted in FIG. 7 as separate components, both the display system 710 and the master assembly 706 may be part of the same device and/or operation control system (e.g., a display device that includes a touchscreen).

In some examples, the medical instrument 704 may include a visualization system, which may include an image capture assembly that records a concurrent or real-time image of a procedural site and provides the image to the operator O through one or more displays of display system 710. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 704. Additionally or alternatively, a separate endoscope, attached to a separate manipulator assembly, may be used with medical instrument 704 to image the procedural site. The visualization system may be implemented as hardware, firmware, software or a combination thereof which interact with or are otherwise executed by one or more computer processors, such as of the control system 712.

Display system 710 may also display an image of the procedural site and medical instruments, which may be captured by the visualization system. In some examples, the medical system 700 provides a perception of telepresence to the operator O. For example, images captured by an imaging device at a distal portion of the medical instrument 704 may be presented by the display system 710 to provide the perception of being at the distal portion of the medical instrument 704 to the operator O. The input to the master assembly 706 provided by the operator O may move the distal portion of the medical instrument 704 in a manner that corresponds with the nature of the input (e.g., distal tip turns right when a trackball is rolled to the right) and results in corresponding change to the perspective of the images captured by the imaging device at the distal portion of the medical instrument 704. As such, the perception of telepresence for the operator O is maintained as the medical instrument 704 is moved using the master assembly 706. The operator O can manipulate the medical instrument 704 and hand controls of the master assembly 706 as if viewing the workspace in substantially true presence, simulating the experience of an operator that is physically manipulating the medical instrument 704 from within the patient anatomy.

In some examples, the display system 710 may present virtual images of a procedural site that are created using image data recorded pre-operatively (e.g., prior to the procedure performed by the medical instrument system 800) or intra-operatively (e.g., concurrent with the procedure performed by the medical instrument system 800), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 710 may display a virtual image that is generated based on tracking the location of medical instrument 704. For example, the tracked location of the medical instrument 704 may be registered (e.g., dynamically referenced) with the model generated using the pre-operative or intra-operative images, with different portions of the model correspond with different locations of the patient anatomy. As the medical instrument 704 moves through the patient anatomy, the registration is used to determine portions of the model corresponding with the location and/or perspective of the medical instrument 704 and virtual images are generated using the determined portions of the model. This may be done to present the operator O with virtual images of the internal procedural site from viewpoints of medical instrument 704 that correspond with the tracked locations of the medical instrument 704.

The medical system 700 may also include the control system 712, which may include processing circuitry that implements the some or all of the methods or functionality discussed herein. The control system 712 may include at least one memory and at least one processor for controlling the operations of the manipulator assembly 702, the medical instrument 704, the master assembly 706, the sensor system 708, and/or the display system 710. Control system 712 may include instructions (e.g., a non-transitory machine-readable medium storing the instructions) that when executed by the at least one processor, configures the one or more processors to implement some or all of the methods or functionality discussed herein. While the control system 712 is shown as a single block in FIG. 7, the control system 712 may include two or more separate data processing circuits with one portion of the processing being performed at the manipulator assembly 702, another portion of the processing being performed at the master assembly 706, and/or the like. In some examples, the control system 712 may include other types of processing circuitry, such as application-specific integrated circuits (ASICs) and/or field-programmable gate array (FPGAs). The control system 712 may be implemented using hardware, firmware, software, or a combination thereof.

In some examples, the control system 712 may receive feedback from the medical instrument 704, such as force and/or torque feedback. Responsive to the feedback, the control system 712 may transmit signals to the master assembly 706. In some examples, the control system 712 may transmit signals instructing one or more actuators of the manipulator assembly 702 to move the medical instrument 704. In some examples, the control system 712 may transmit informational displays regarding the feedback to the display system 710 for presentation or perform other types of actions based on the feedback.

The control system 712 may include a virtual visualization system to provide navigation assistance to operator O when controlling the medical instrument 704 during an image-guided medical procedure. Virtual navigation using the virtual visualization system may be based upon an acquired pre-operative or intra-operative dataset of anatomic passageways of the patient P. The control system 712 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 708 that is used to compute an (e.g., approximate) location of the medical instrument 704 with respect to the anatomy of patient P. The sensor system 708 may be used to register and display the medical instrument 704 together with the pre-operatively or intra-operatively recorded images. For example, PCT Publication WO 2016/161298 (published Dec. 1, 2016 and titled “Systems and Methods of Registration for Image Guided Surgery”), which is incorporated by reference herein in its entirety, discloses example systems.

During a virtual navigation procedure, the sensor system 708 may be used to compute the (e.g., approximate) location of the medical instrument 704 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,300,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 700 may further include operations and support systems (not shown) such as illumination systems, steering control systems, irrigation systems, and/or suction systems. In some examples, the medical system 700 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

FIG. 8A is a simplified diagram of a medical instrument system 800 according to some examples. The medical instrument system 800 includes a flexible elongate device 802 (also referred to as elongate device 802), a drive unit 804, and a medical tool 826 that collectively is an example of a medical instrument 704 of a medical system 700. The medical system 700 may be a teleoperated system, a non-teleoperated system, or a hybrid teleoperated and non-teleoperated system, as explained with reference to FIG. 7. A visualization system 831, tracking system 830, and navigation system 832 are also shown in FIG. 8A and are example components of the control system 712 of the medical system 700. In some examples, the medical instrument system 800 may be used for non-teleoperational exploratory procedures or in procedures involving traditional manually operated medical instruments, such as endoscopy. The medical instrument system 800 may be used to gather (e.g., measure) a set of data points corresponding to locations within anatomic passageways of a patient, such as patient P.

The elongate device 802 is coupled to the drive unit 804. The elongate device 802 includes a channel 821 through which the medical tool 826 may be inserted. The elongate device 802 navigates within patient anatomy to deliver the medical tool 826 to a procedural site. The elongate device 802 includes a flexible body 816 having a proximal end 817 and a distal end 818. In some examples, the flexible body 816 may have an approximately 3 mm outer diameter. Other flexible body outer diameters may be larger or smaller.

Medical instrument system 800 may include the tracking system 830 for determining the position, orientation, speed, velocity, pose, and/or shape of the flexible body 816 at the distal end 818 and/or of one or more segments 824 along flexible body 816, as will be described in further detail below. The tracking system 830 may include one or more sensors and/or imaging devices. The flexible body 816, such as the length between the distal end 818 and the proximal end 817, may include multiple segments 824. The tracking system 830 may be implemented using hardware, firmware, software, or a combination thereof. In some examples, the tracking system 830 is part of control system 712 shown in FIG. 7.

Tracking system 830 may track the distal end 818 and/or one or more of the segments 824 of the flexible body 816 using a shape sensor 822. The shape sensor 822 may include an optical fiber aligned with the flexible body 816 (e.g., provided within an interior channel of the flexibly body 816 or mounted externally along the flexible body 816). In some examples, the optical fiber may have a diameter of approximately 800 μm. In other examples, the diameter may be larger or smaller. The optical fiber of the shape sensor 822 may form a fiber optic bend sensor for determining the shape of flexible body 816. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some examples, are described in U.S. Patent Application Publication No. 8006/0013523 (filed Jul. 13, 8005 and titled “Fiber optic position and shape sensing device and method relating thereto”); U.S. Pat. No. 7,772,541 (filed on Mar. 12, 8008 and titled “Fiber Optic Position and/or Shape Sensing Based on Rayleigh Scatter”); and U.S. Pat. No. 8,773,350 (filed on Sept. 2, 2010 and titled “Optical Position and/or Shape Sensing”), which are all incorporated by reference herein in their entireties. Sensors in some examples may employ other suitable strain sensing techniques, such as Rayleigh scattering, Raman scattering, Brillouin scattering, and Fluorescence scattering.

In some examples, the shape of the flexible body 816 may be determined using other techniques. For example, a history of the position and/or pose of the distal end 818 of the flexible body 816 can be used to reconstruct the shape of flexible body 816 over an interval of time (e.g., as the flexible body 816 is advanced or retracted within a patient anatomy). In some examples, the tracking system 830 may alternatively and/or additionally track the distal end 818 of the flexible body 816 using a position sensor system 820. Position sensor system 820 may be a component of an EM sensor system with the position sensor system 820 including one or more position sensors. Although the position sensor system 820 is shown as being near the distal end 818 of the flexible body 816 to track the distal end 818, the number and location of the position sensors of the position sensor system 820 may vary to track different regions along the flexible body 816. In one example, the position sensors include conductive coils that may be subjected to an externally generated electromagnetic field. Each coil of position sensor system 820 may produce an induced electrical signal having characteristics that depend on the position and orientation of the coil relative to the externally generated electromagnetic field. The position sensor system 820 may measure one or more position coordinates and/or one or more orientation angles associated with one or more portions of flexible body 816. In some examples, the position sensor system 820 may be configured and positioned to measure six degrees of freedom, e.g., three position coordinates X, Y, Z and three orientation angles indicating pitch, yaw, and roll of a base point. In some examples, the position sensor system 820 may be configured and positioned to measure five degrees of freedom, e.g., three position coordinates X, Y, Z and two orientation angles indicating pitch and yaw of a base point. Further description of a position sensor system, which may be applicable in some examples, is provided in U.S. Pat. No. 6,380,432 (filed Aug. 11, 1999 and titled “Six-Degree of Freedom Tracking System Having a Passive Transponder on the Object Being Tracked”), which is incorporated by reference herein in its entirety.

In some examples, the tracking system 830 may alternately and/or additionally rely on a collection of pose, position, and/or orientation data stored for a point of an elongate device 802 and/or medical tool 826 captured during one or more cycles of alternating motion, such as breathing. This stored data may be used to develop shape information about the flexible body 816. In some examples, a series of position sensors (not shown), such as EM sensors like the sensors in position sensor 820 or some other type of position sensors may be positioned along the flexible body 816 and used for shape sensing. In some examples, a history of data from one or more of these position sensors taken during a procedure may be used to represent the shape of elongate device 802, particularly if an anatomic passageway is generally static.

FIG. 8B is a simplified diagram of the medical tool 826 within the elongate device 802 according to some examples. The flexible body 816 of the elongate device 802 may include the channel 821 sized and shaped to receive the medical tool 826. In some examples, the medical tool 826 may be used for procedures such as diagnostics, imaging, surgery, biopsy, ablation, illumination, irrigation, suction, electroporation, etc. Medical tool 826 can be deployed through channel 821 of flexible body 816 and operated at a procedural site within the anatomy. Medical tool 826 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 826 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 826 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 821 when the biopsy tool is within the channel 821. The medical tool 826 may be an image capture probe that includes a distal portion with a stereoscopic or monoscopic camera that may be placed at or near the distal end 818 of flexible body 816 for capturing images (e.g., still or video images). The captured images may be processed by the visualization system 831 for display and/or provided to the tracking system 830 to support tracking of the distal end 818 of the flexible body 816 and/or one or more of the segments 824 of the flexible body 816. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 831. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 816 of the elongate device 802 to facilitate visual navigation of the elongate device 802 to a procedural site and then is replaced within the flexible body 816 with another type of medical tool 826 that performs the procedure. In some examples, the image capture probe may be within the flexible body 816 of the elongate device 802 along with another type of medical tool 826 to facilitate simultaneous image capture and tissue intervention, such as within the same channel 821 or in separate channels. A medical tool 826 may be advanced from the opening of the channel 821 to perform the procedure (or some other functionality) and then retracted back into the channel 821 when the procedure is complete. The medical tool 826 may be removed from the proximal end 817 of the flexible body 816 or from another optional instrument port (not shown) along flexible body 816.

In some examples, the elongate device 802 may include integrated imaging capability rather than utilize a removable image capture probe. For example, the imaging device (or fiber-optic bundle) and the light emitters may be located at the distal end 818 of the elongate device 802. The flexible body 215 may include one or more dedicated channels that carry the cable(s) and/or optical fiber(s) between the distal end 818 and the visualization system 831. Here, the medical instrument system 800 can perform simultaneous imaging and tool operations.

In some examples, the medical tool 826 is capable of controllable articulation. The medical tool 826 may house cables (which may also be referred to as pull wires), linkages, or other actuation controls (not shown) that extend between its proximal and distal ends to controllably bend the distal end of medical tool 826, such as discussed herein for the flexible elongate device 802. The medical tool 826 may be coupled to a drive unit 804 and the manipulator assembly 702. In these examples, the elongate device 802 may be excluded from the medical instrument system 800 or may be a flexible device that does not have controllable articulation. Steerable instruments or tools, applicable in some examples, are further described in detail in U.S. Pat. No. 7,916,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sept. 30, 2008 and titled “Passive Preload and Capstan Drive for Surgical Instruments”), which are incorporated by reference herein in their entireties.

The flexible body 816 of the elongate device 802 may also or alternatively house cables, linkages, or other steering controls (not shown) that extend between the drive unit 804 and the distal end 818 to controllably bend the distal end 818 as shown, for example, by broken dashed line depictions 819 of the distal end 818 in FIG. 8A. In some examples, at least four cables are used to provide independent up-down steering to control a pitch of the distal end 818 and left-right steering to control a yaw of the distal end 281. In these examples, the flexible elongate device 802 may be a steerable catheter. Examples of steerable catheters, applicable in some examples, are described in detail in PCT Publication WO 2019/018436 (published Jan. 24, 2019 and titled “Flexible Elongate Device Systems and Methods”), which is incorporated by reference herein in its entirety.

In examples where the elongate device 802 and/or medical tool 826 are actuated by a teleoperational assembly (e.g., the manipulator assembly 702), the drive unit 804 may include drive inputs that removably couple to and receive power from drive elements, such as actuators, of the teleoperational assembly. In some examples, the elongate device 802 and/or medical tool 826 may include gripping features, manual actuators, or other components for manually controlling the motion of the elongate device 802 and/or medical tool 826. The elongate device 802 may be steerable or, alternatively, the elongate device 802 may be non-steerable with no integrated mechanism for operator control of the bending of distal end 818. In some examples, one or more channels 821 (which may also be referred to as lumens), through which medical tools 826 can be deployed and used at a target anatomical location, may be defined by the interior walls of the flexible body 816 of the elongate device 802.

In some examples, the medical instrument system 800 (e.g., the elongate device 802 or medical tool 826) may include a flexible bronchial instrument, such as a bronchoscope or bronchial catheter, for use in examination, diagnosis, biopsy, and/or treatment of a lung. The medical instrument system 800 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 830 may be sent to the navigation system 832, where the information may be combined with information from the visualization system 831 and/or pre-operatively obtained models to provide the physician, clinician, surgeon, or other operator with real-time position information. In some examples, the real-time position information may be displayed on the display system 710 for use in the control of the medical instrument system 800. In some examples, the navigation system 832 may utilize the position information as feedback for positioning medical instrument system 800. Various systems for using fiber optic sensors to register and display a surgical instrument with surgical images, applicable in some examples, are provided in U.S. Pat. No. 8,300,131 (filed May 13, 2011 and titled “Medical System Providing Dynamic Registration of a Model of an Anatomic Structure for Image-Guided Surgery”), which is incorporated by reference herein in its entirety.

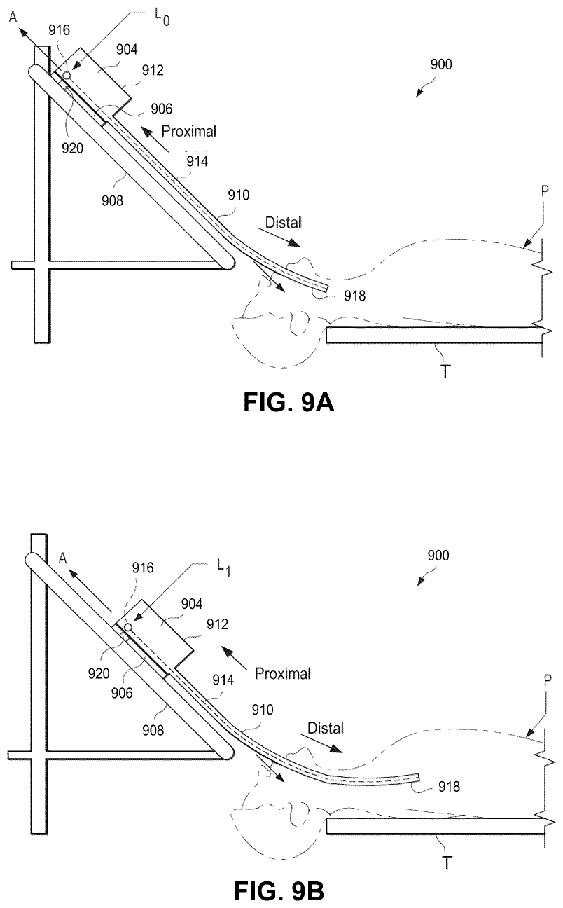

FIGS. 9A and 9B are simplified diagrams of side views of a patient coordinate space including a medical instrument mounted on an insertion assembly according to some examples. As shown in FIGS. 9A and 9B, a surgical environment 900 may include a patient P positioned on the patient table T. Patient P may be stationary within the surgical environment 900 in the sense that gross patient movement is limited by sedation, restraint, and/or other means. Cyclic anatomic motion, including respiration and cardiac motion, of patient P may continue. Within surgical environment 900, a medical instrument 904 is used to perform a medical procedure which may include, for example, surgery, biopsy, ablation, illumination, irrigation, suction, or electroporation. The medical instrument 904 may also be used to perform other types of procedures, such as a registration procedure to associate the position, orientation, and/or pose data captured by the sensor system 708 to a desired (e.g., anatomical or system) reference frame. The medical instrument 904 may be, for example, the medical instrument 704. In some examples, the medical instrument 904 may include an elongate device 910 (e.g., a catheter) coupled to an instrument body 912. Elongate device 910 includes one or more channels sized and shaped to receive a medical tool.

Elongate device 910 may also include one or more sensors (e.g., components of the sensor system 708). In some examples, a shape sensor 914 may be fixed at a proximal point 916 on the instrument body 912. The proximal point 916 of the shape sensor 914 may be movable with the instrument body 912, and the location of the proximal point 916 with respect to a desired reference frame may be known (e.g., via a tracking sensor or other tracking device). The shape sensor 914 may measure a shape from the proximal point 916 to another point, such as a distal end 918 of the elongate device 910. The shape sensor 914 may be aligned with the elongate device 910 (e.g., provided within an interior channel or mounted externally). In some examples, the shape sensor 914 may use optical fibers to generate shape information for the elongate device 910.

In some examples, position sensors (e.g., EM sensors) may be incorporated into the medical instrument 904. A series of position sensors may be positioned along the flexible elongate device 910 and used for shape sensing. Position sensors may be used alternatively to the shape sensor 914 or with the shape sensor 914, such as to improve the accuracy of shape sensing or to verify shape information.

Elongate device 910 may house cables, linkages, or other steering controls that extend between the instrument body 912 and the distal end 918 to controllably bend the distal end 918. In some examples, at least four cables are used to provide independent up-down steering to control a pitch of distal end 918 and left-right steering to control a yaw of distal end 918. The instrument body 912 may include drive inputs that removably couple to and receive power from drive elements, such as actuators, of a manipulator assembly.

The instrument body 912 may be coupled to an instrument carriage 906. The instrument carriage 906 may be mounted to an insertion stage 908 that is fixed within the surgical environment 900. Alternatively, the insertion stage 908 may be movable but have a known location (e.g., via a tracking sensor or other tracking device) within surgical environment 900. Instrument carriage 906 may be a component of a manipulator assembly (e.g., manipulator assembly 702) that couples to the medical instrument 904 to control insertion motion (e.g., motion along an insertion axis A) and/or motion of the distal end 918 of the elongate device 910 in multiple directions, such as yaw, pitch, and/or roll. The instrument carriage 906 or insertion stage 908 may include actuators, such as servomotors, that control motion of instrument carriage 906 along the insertion stage 908.

A sensor device 920, which may be a component of the sensor system 708, may provide information about the position of the instrument body 912 as it moves relative to the insertion stage 908 along the insertion axis A. The sensor device 920 may include one or more resolvers, encoders, potentiometers, and/or other sensors that measure the rotation and/or orientation of the actuators controlling the motion of the instrument carriage 906, thus indicating the motion of the instrument body 912. In some examples, the insertion stage 908 has a linear track as shown in FIGS. 9A and 9B. In some examples, the insertion stage 908 may have curved track or have a combination of curved and linear track sections.

FIG. 9A shows the instrument body 912 and the instrument carriage 906 in a retracted position along the insertion stage 908. In this retracted position, the proximal point 916 is at a position LO on the insertion axis A. The location of the proximal point 916 may be set to a zero value and/or other reference value to provide a base reference (e.g., corresponding to the origin of a desired reference frame) to describe the position of the instrument carriage 906 along the insertion stage 908. In the retracted position, the distal end 918 of the elongate device 910 may be positioned just inside an entry orifice of patient P. Also in the retracted position, the data captured by the sensor device 920 may be set to a zero value and/or other reference value (e.g., I=0). In FIG. 9B, the instrument body 912 and the instrument carriage 906 have advanced along the linear track of insertion stage 908, and the distal end 918 of the elongate device 910 has advanced into patient P. In this advanced position, the proximal point 916 is at a position L1 on the insertion axis A. In some examples, the rotation and/or orientation of the actuators measured by the sensor device 920 indicating movement of the instrument carriage 906 along the insertion stage 908 and/or one or more position sensors associated with instrument carriage 906 and/or the insertion stage 908 may be used to determine the position L1 of the proximal point 916 relative to the position LO. In some examples, the position L1 may further be used as an indicator of the distance or insertion depth to which the distal end 918 of the elongate device 910 is inserted into the passageway(s) of the anatomy of patient P.

As noted above, in some implementations, any of the methods or techniques described above with reference to FIGS. 1-6 may be performed by the medical system 700 or components/subsystems thereof. For example, the control system 712 may perform any processing, calculations, and/or determinations described above with reference to FIGS. 1-6, the sensor system 708 may perform (or be used to perform) any sensing or detecting operations described above with reference to FIGS. 1-6, and/or the control system 712 and/or visualization system 831 may cause the display system 710 to display and/or modify any GUI described above with reference to FIGS. 1-6 (e.g., GUI 100). In examples where a GUI (e.g., GUI 100) provides both input and output capability, the display system 710 and master assembly 706 may be at least partially integrated (e.g., include a touchscreen). The flexible elongate device and the treatment tool referenced above in connection with any of FIGS. 1-6 may be the flexible elongate device 802 and the medical tool 826, respectively, of FIG. 8A and/or FIG. 8B.

One or more components of the examples discussed in this disclosure, such as control system 712, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 502.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain examples and examples have been described above and shown in the accompanying drawings, it is to be understood that such examples and examples are merely illustrative and are not limited to the specific constructions and arrangements shown and described, since various other alternatives, modifications, and equivalents will be appreciated by those with ordinary skill in the art.

1. A method for planning, or navigating during, a medical procedure, the method comprising:
obtaining, by one or more processors, a model representing an internal anatomy of a patient and a target lesion;
determining, by the one or more processors and based on a pose of a flexible elongate device, an expected tool trajectory of a treatment tool extendable from the flexible elongate device;
determining, by the one or more processors and based on the expected tool trajectory and an insertion distance of the treatment tool, a projected treatment zone in relation to the model; and
causing, by the one or more processors, a display device to display a graphical user interface depicting (i) the expected tool trajectory, and (ii) the projected treatment zone, in relation to the model.

2. The method of claim 1, wherein the insertion distance comprises a virtual insertion distance indicated by a user.

3. (canceled)

4. (canceled)

5. The method of claim 29 , wherein the graphical user interface depicts the expected tool trajectory as a path that extends beyond the virtual insertion distance, and wherein the method further comprises:
causing, by the one or more processors, the projected treatment zone to move along the path on the graphical user interface in response to user input via a virtual insertion distance control.

6. (canceled)

7. The method of claim 1, wherein the flexible elongate device comprises an actual flexible elongate device, the method further comprising:
determining the pose of the flexible elongate device based on shape sensor data generated using a shape sensor of the actual flexible elongate device.

8. (canceled)

9. (canceled)

10. (canceled)

11. The method of claim 7, further comprising:
determining, by the one or more processors, the insertion distance using an insertion sensor of the flexible elongate device.

12. The method of claim 7, further comprising:
causing, by the one or more processors and in response to detecting that a treatment is performed at a treatment site, the graphical user interface to depict a completed treatment zone at the treatment site in relation to the model.

13. The method of claim 12, further comprising:
determining, by the one or more processors, one or more of a size or shape of the completed treatment zone based on one or more of (i) a power or duration associated with the treatment (ii) proximity of the treatment site to one or more critical structures of the patient, (iii) one or more tissue characteristics of the patient at the treatment site, or (iv) one or more of an actual size or shape associated with the treatment at the treatment site.

14. (canceled)

15. (canceled)

16. The method of claim 1, wherein the flexible elongate device comprises a virtual flexible elongate device, and wherein the method further comprises:
causing, by the one or more processors, the graphical user interface to depict a planned pose of the virtual flexible elongate device.

17. The method of claim 1, further comprising:
in response to user input, causing, by the one or more processors, the graphical user interface to change the projected treatment zone to a marked treatment zone that has a fixed position irrespective of further adjustments to the insertion distance.

18. (canceled)

19. (canceled)

20. (canceled)

21. The method of claim 17, wherein the insertion distance comprises a virtual insertion distance indicated by the user via a virtual insertion distance control on the graphical user interface, and wherein the method further comprises:
in response to user manipulation of the virtual insertion distance control occurring after the projected treatment zone is changed to the marked treatment zone, causing, by the one or more processors, the graphical user interface to display a new treatment zone in relation to the model.

22. (canceled)

23. The method of claim 1, wherein the projected treatment zone comprises an ellipsoid surrounding a position located at the insertion distance along the expected tool trajectory.

24. The method of claim 1, further comprising:
determining, by the one or more processors, a major axis of the target lesion; and
causing, by the one or more processors, the graphical user interface to depict the major axis of the target lesion.

25. (canceled)

26. (canceled)

27. The method of claim 1, further comprising:
causing, by the one or more processors, the graphical user interface to adjust one or more of a size or shape of the projected treatment zone based on proximity of the projected treatment zone to one or more structures within the patient.

28. (canceled)

29. (canceled)

30. The method of claim 17, further comprising:
in response to the user input, setting, by the one or more processors, one or more of a power parameter or a duration parameter of medical equipment to a value that corresponds to one or more of an adjusted size or shape of the projected treatment zone.

31. (canceled)

32. The method of claim 1, further comprising:
determining, by the one or more processors, a suggested probe trajectory for a next treatment site; and
causing, by the one or more processors, the graphical user interface to depict the suggested probe trajectory.

33.-41. (canceled)

42. A system comprising:
one or more processors;
a display device; and
one or more non-transitory, computer-readable media storing instructions that, when executed by the one or more processors, cause the one or more processors to:
obtain a model representing an internal anatomy of a patient and a target lesion;
determine, based on a pose of a flexible elongate device, an expected tool trajectory of a treatment tool extendable from the flexible elongate device;
determine, based on the expected tool trajectory and an insertion distance of the treatment tool, a projected treatment zone in relation to the model; and
cause a display device to display a graphical user interface depicting (i) the expected tool trajectory, and (ii) the projected treatment zone, in relation to the model.

43.-45. (canceled)

46. A method for planning, or navigating during, a medical procedure, the method comprising:
obtaining, by one or more processors, a model representing a target lesion within a patient;
determining, by the one or more processors, a position of a flexible elongate device in relation to the model; and
determining, by the one or more processors and based on a position of the target lesion and the position of the flexible elongate device, a plurality of trajectories along which a treatment tool extendable from the flexible elongate device can approach the target lesion to collectively provide treatment coverage across at least one dimension of the target lesion.

47.-75. (canceled)

76. A system comprising:
one or more processors; and
one or more non-transitory, computer-readable media storing instructions that, when executed by the one or more processors, cause the one or more processors to:
obtain a model representing a target lesion within a patient;
determine a position of a flexible elongate device in relation to the model; and
determine, based on a position of the target lesion and the position of the flexible elongate device, a plurality of trajectories along which a treatment tool extendable from the flexible elongate device can approach the target lesion to collectively provide treatment coverage across at least one dimension of the target lesion.

77.-79. (canceled)

80. The system of claim 42, wherein the graphical user interface depicts the expected tool trajectory as a path that extends beyond a virtual insertion distance indicated by a user, and wherein the instructions, when executed, cause the one or more processors to:
cause the projected treatment zone to move along the path on the graphical user interface in response to user input via a virtual insertion distance control.

81. The system of claim 42, wherein the instructions, when executed, cause the one or more processors to:
in response to detecting that a treatment is performed at a treatment site, cause the graphical user interface to depict a completed treatment zone at the treatment site in relation to the model
